(19) United States
(12) Patent Application Publication
(10) Pub. No.: US 2006/0155379 A1
(43) Pub. Date: Jul. 13, 2006

(54) Expandable implant for repairing a defect in a nucleus of an intervertebral disc

(76) Inventors: Scott H. Heneveld SR., Whitmore, CA (US); James C. Thomas JR., Las Vegas, NV (US)

Correspondence Address: CHRISTIE, PARKER & HALE, LLP, PO BOX 7068, PASADENA, CA, 91109-7068, US

(21) Appl. No.: 11/258,678

(22) Filed: Oct. 25, 2005

Related U.S. Application Data
(60) Provisional application No. 60/621,305, filed on Oct. 25, 2004.
(60) Provisional application No. 60/645,192, filed on Jan. 21, 2005.
(60) Provisional application No. 60/667,031, filed on Apr. 1, 2005.

(51) Int. Cl. A61F 2/44; A61F 2/46

(52) U.S. Cl. 623/17.16

(57) ABSTRACT

Expandable implants for repairing a nuclear defect of an intervertebral disc, and methods and apparatuses for delivering the same into the disc. The implants generally have a compressed form having a size adapted for insertion into the intervertebral disc, and a composition that allows the implant to expand from the compressed form into an expanded form after the implant is inserted into the nucleus. The expanded form of the implant has a configuration that fills the nuclear defect. The composition used to make the implant can include a shape memory alloy (SMA) or any other suitable material. Various devices can be used to insert the implants into the area being treated.

### CROSS-REFERENCE TO RELATED APPLICATIONS

This application claims the benefit of U.S. Provisional Application No. 60/621,305 filed on Oct. 25, 2004, U.S. Provisional Application No. 60/645,192 filed on Jan. 21, 2005, and U.S. Provisional Application No. 60/667,031 filed on Apr. 1, 2005.

### FIELD OF THE INVENTION

The present invention relates to expandable implants for repairing a defect in a nucleus of an intervertebral disc.

### BACKGROUND OF THE INVENTION

A lumbar intervertebral disc comprises a mechanical and flexible component of the spine to allow better support of the vertebral body and the spinal column. The disc is made of two components, an annulus and a nucleus. The annulus is the outer structure and is composed of multiple layers of collagen fibers. Each fiber is uniquely oriented at 30 degrees to the adjacent fiber. When intact the intervertebral disc can support pressures of up to 400 lbs. due to its hydrostatic nature. The nucleus is the inner structure and is composed of a different collagen, which is largely water and in a gelatinous form. The nucleus is held under pressure in the center of the intact disc by the intact annulus (see FIGS. 1a and 1b). Unfortunately, the annulus is prone to tears and traumatic events. When a tear occurs from the periphery of the annulus to the center of the nucleus, this comprises a radial annular tear. The disc's ability to act in a hydrostatic fashion is substantially compromised. This will allow the nucleus to rupture through the annular tear into and towards the spinal canal (see FIGS. 2a & 2b). This ruptured nucleus material puts pressure on the neural and ligamentous structures causing back pain and often pain down the posterior aspect of the buttock and leg. This particular symptom is referred to as sciatica.

Conservative, non-surgical treatment is often performed. However, when such treatment fails and pain is intractable or neurologic deficit exists, surgery is performed. In one type of surgery, a small opening (a laminotomy) is made in the back of the spinal bone structure to allow access to the spinal canal. The nerve root and thecal sac are gently retracted and the hernia identified. The hernia is essentially removed with micro surgical tools and instruments. A defect is left in the annulus, and rather than placing an implant or object in the annular defect, the patient relies on a fibroblastic response to repair the defect with scar tissue.

However, the vascularity of the adult intervertebral disc is poor. The disc is the largest avascular structure in the human body next to the cornea of the eye. As a result, healing with scar tissue is very fragile, if it occurs at all, and often, over a period of years, further degeneration of the annular and nuclear structures occurs. The hydrostatic property is not restored. The disc space often narrows as a result of this progressive degeneration, and this causes new problems such as root compression in the exit zone of the spinal canal. This area is known as the foramen. This may result in the patient having increased or recurrent symptoms, and a subsequent surgical operation may be required for the patient. The statistics vary for the number of patients who have laminectomy and discectomy and subsequently require fusion. They may be as high as 70% over a ten year period.

In addition to the problems that exist with the repair of annular defects, the same obstacles have been present with respect to nuclear defects. Because the nucleus often ruptures through tears in the annulus, there often is an inadequate amount of residual nucleus for the disc to provide its weight bearing support and compression functions. As a result, there exists a need for an implant that can be inserted into the nucleus to attempt to simulate the function and structure of the original disc. Nucleus replacement implants have been developed to simulate the original nucleus. The most popular attempts have utilized hydrogel configurations. Migration after implantation has been a concern with these types of implants. The ability to restore and maintain disc space height is also lacking in many of these types of implants.

### SUMMARY OF THE INVENTION

The present invention relates to expandable implants for replacing the nucleus of an intervertebral disc and methods and apparatuses for delivering the same into the disc. In one embodiment, the implants generally comprise a compressed form having a size adapted for insertion via a cannula into the intervertebral disc, and a composition that allows the implant to expand from the compressed form into an expanded form after the implant is inserted into the nuclear cavity. The cavity is created by resection of the nucleus via various forms, such as manually in an open or percutaneous fashion, or chemically dissolved with chemicals or hydrolysis, or vaporized with radio frequency and/or laser engery. The expanded form of the implant has a configuration that fills the nuclear defect. The composition used to make the implant can comprise a shape memory alloy (SMA) or any other suitable materials. The implant will ideally replace hydrostatic load capacity with a mechanical or functional spring within the intervertebral disc. The defect is the residual state in the nucleus after nuclear resection.

Various devices can be used to insert the present implants into the area being treated. The devices are adapted to retain the implant while the device is inserted into the intervertebral disc, and to controllably release the implant therein.

### BRIEF DESCRIPTION OF THE DRAWINGS

FIG. 1a shows an axial view of a normal disc and the spinal cord;

FIG. 1b shows a side view of a normal disc and the spinal cord;

FIG. 2a shows an axial view of a ruptured disc putting pressure on the spinal cord;

FIG. 2b shows a side view of a ruptured disc putting pressure on the spinal cord;

FIG. 3a shows an axial view of the ruptured disc of FIG. 2a after the herniation has been removed and an annular defect remains;

FIG. 3b shows a side view of the ruptured disc of FIG. 2b after the herniation has been removed and an annular defect remains;

FIG. 4 shows an isometric view of a “nested spiral sheets” configuration of an implant;

FIG. 5 shows an isometric view of an individual spiral sheet in its natural state and in its wound or compressed state;

FIG. 6 shows an isometric view of a “helical wire spring” configuration of an implant shown inserted into the nuclear space;

FIG. 7 shows an isometric view of a “helical ribbon spring” configuration of an implant;

FIG. 8 shows an isometric view of a “combination” configuration of an implant;

FIG. 9 shows an isometric view of the “nested spiral sheets” configuration captured in a pliable pouch;

FIGS. 10a, 10b and 10c show isometric views of a “slit sheet” configuration of an implant being introduced by a cannula delivery device;

FIGS. 11a and 11b show isometric views of the “nitinol collet” configuration of a delivery device;

FIG. 12 shows an isometric view of a “cartridge” configuration delivery system;

FIG. 13a shows an isometric view of a soft edge sheet configuration;

FIG. 13b shows an isometric view of a spiral sheet having a wear prevention barrier;

FIG. 13c shows an isometric view of a “nested spiral sheets” configuration having a wear prevention barrier;

FIG. 14 shows an isometric view of a “nested spiral sheets” configuration with a ratchet mechanism;

FIG. 15 shows a detail view of a dimple feature for a ratchet mechanism;

FIG. 16 shows a flat sheet with the dimple and window features for a ratchet mechanism;

FIG. 17 shows a detail of an interlock for a ratchet mechanism;

FIG. 18 shows an isometric view of sheets nested within each other in a perpendicular orientation;

FIG. 19 shows an assembly sequence for connecting two nitinol sheets; and

FIG. 20 shows a sequence for forming a spiral wound sheet.

### DETAILED DESCRIPTION

The expandable implants of the present invention are suitable for several applications, particularly nuclear defects and damaged intervetebral discs. Several possible configurations can be made from a number of different materials.

The present implants are preferably elastic and susceptible to withstanding long-term implantation into a mammalian body. Examples of suitable materials include shape memory alloys (SMAs), superelastic SMAS, nitinol, MP35, Elgiloy, spring steel, and any plastic elastic material or other material suitable for such implantation. For simplicity and clarity, many of the embodiments described herein are discussed as being made from a SMA, particularly nitinol, but it is understood that the benefits and features of the present invention are not limited to an SMA or nitinol, and can be achieved by using any of other suitable materials.

SMAs are materials that have the ability to return to a predetermined shape. The return is the result of a change of phase or structure that can be triggered by an external stimulus such as temperature change or electrical current. For example, when one type of SMA is below transformation temperature, it has a low yield strength and can be deformed into a new shape that it will retain while it is below its transformation temperature. However, when the material is heated above its transformation temperature, it undergoes a change in crystal structure that causes it to return to its original shape. If the SMA encounters any resistance during this transformation, it can generate extremely large forces. Thus, SMAs provide a good mechanism for remote actuation. One preferred shape memory material is an alloy of nickel and titanium called nitinol. Nitinol has desirable electrical and mechanical properties, a long fatigue life, high corrosion resistance, and has similar properties to residual annular tissue and cartilaginous tissues. Other SMAs can comprise, for example, alloys of copper, zinc and aluminum or copper, aluminum and nickel. For the present invention, SMA materials or a hybrid with SMA materials can be used to make implants to reconstruct the annular and/or nuclear defects after human discectomy surgery, as well as a variety of bone fractures experienced throughout the human body.

Another type of shape memory alloy is called superelastic SMAs, which can be compressed into a small shape and upon release can automatically expand to a predetermined shape. Thus, no external activation, such as temperature or electrical stimulation, is required. One preferred superelastic SMA is superelastic nitinol, which has similar properties to the SMA nitinol discussed above, but because it is a superelastic SMA does not require activation. The superelastic nitinol, or other suitable superelastic SMA, can be compressed into a small package, placed into a surgical deficit such as an annular or nuclear defect or bone fracture and, upon release, expand to a predetermined shape to fill the deficit.

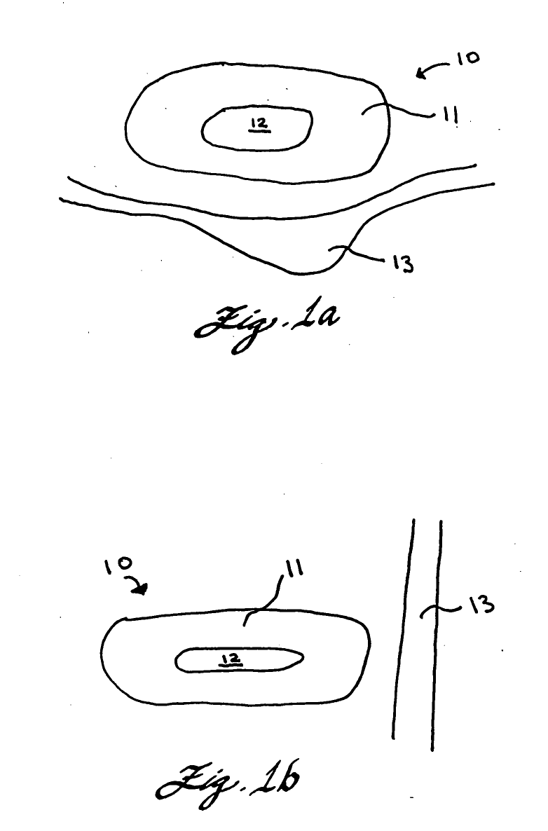

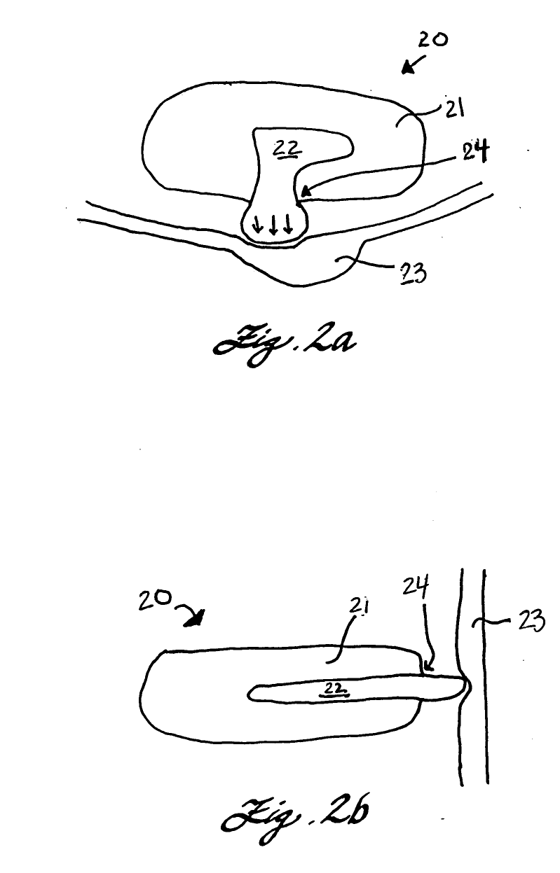

The implants of the present invention are advantageous for treatment of nuclear defects. The implants can be made from materials such as nitinol and are inserted into the nuclear cavity to replace the resected nucleus and augment residual nuclear function, and hopefully to restore weight bearing support to the intervertebral disc. FIGS. 1 to 3 illustrate a normal disc, a ruptured disc, and a disc that has undergone a discectomy.

Referring to FIG. 1a, an axial view of a normal, unruptured disc 10 is shown. The disc 10 comprises an annulus 11 surrounding a nucleus 12. The spinal cord or nerve 13 is shown in close proximity to the disc, but no portion of the disc is putting pressure on the nerve. FIG. 1b shows a side view of the disc 10 of FIG. 1a.

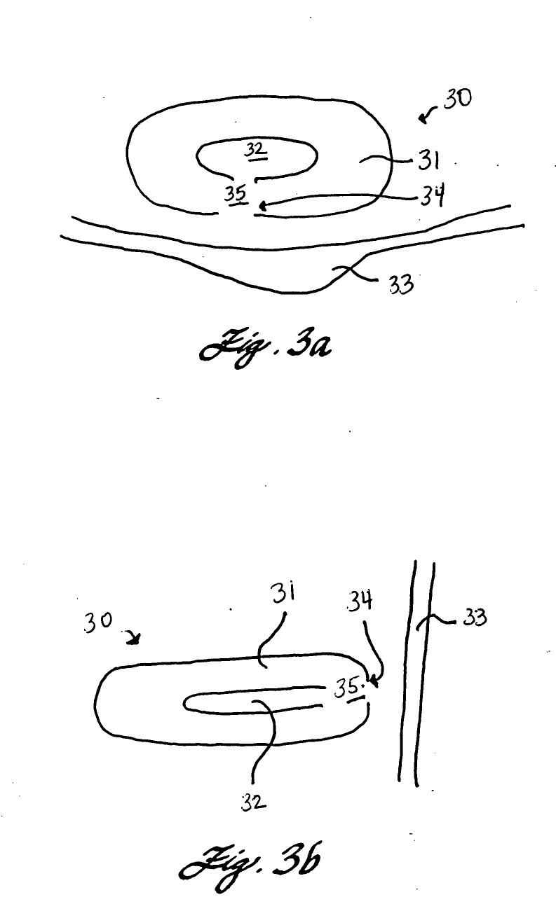

Referring to FIG. 2a, an axial view of a ruptured, herniated disc 20 is shown. The annulus 21 has suffered an annular tear 24, which allowed a portion of the nucleus 22 to rupture through the annulus and put pressure on the nerve 23 (i.e. sciatica). FIG. 2b shows a side view of the ruptured disc 20 of FIG. 2a.

Referring to FIG. 3a, an axial view is shown of the disc 30 after a partial discectomy has been performed to remove the hernia. After the hernia has been removed, the annular tear 34 is still present, but rather than having the portion of the nucleus ruptured through the annulus 31, there remains an annular defect 35, which in effect is an empty space. As noted above, because the nucleus often ruptures through tears in the annulus, there often is an inadequate amount of residual nucleus for the disc to provide weight bearing support and resist compression in the various anatomical positions of sitting or standing. As also noted above, a common practice is to use nucleus replacement implants consisting of hydrogel or other materials having similar properties. FIG. 3b shows a side view of the disc 30 of FIG. 3a.

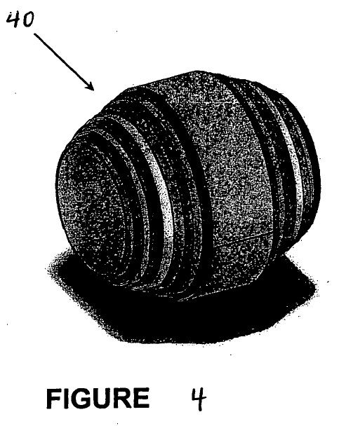

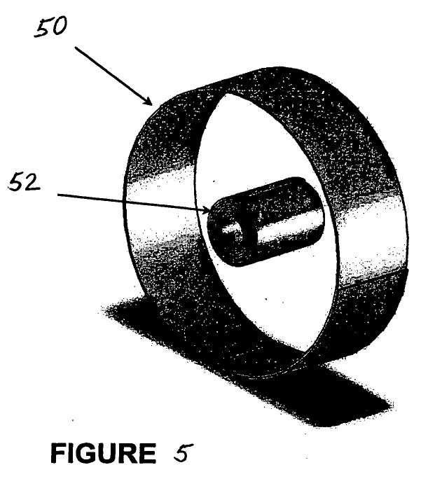

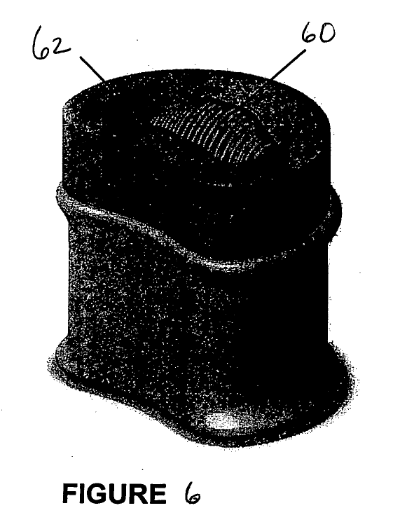

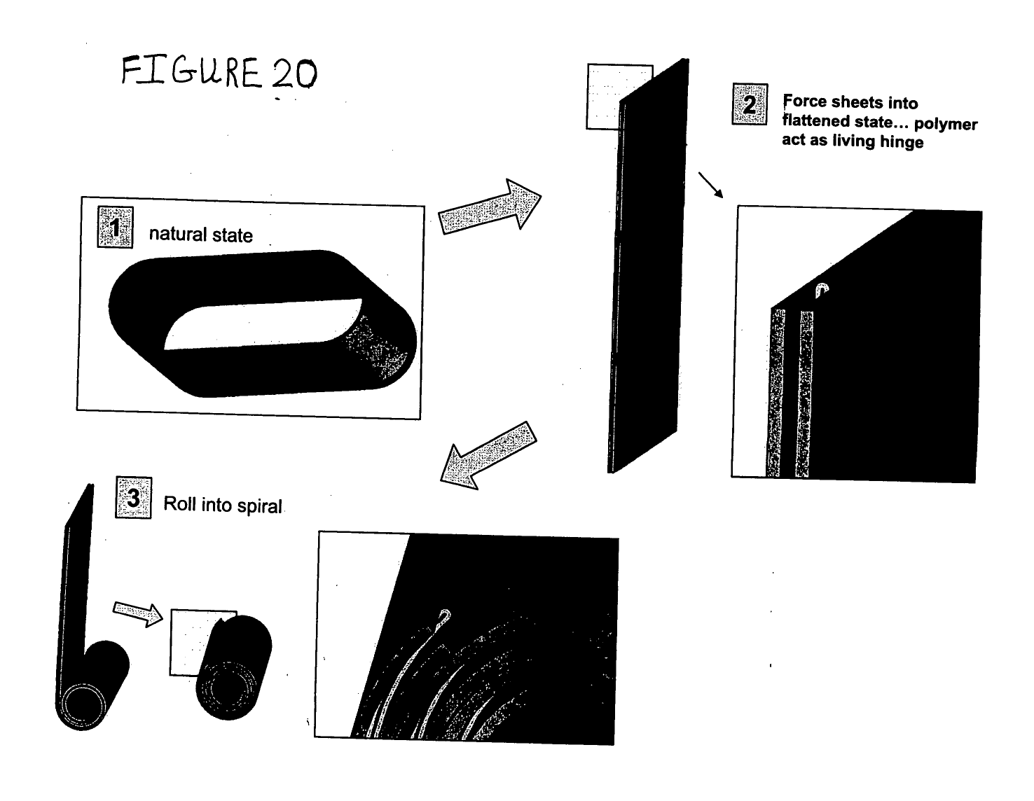

An exemplary application of the present implants involves replacing or augmenting the nucleus of the disc. As shown in FIG. 4, one exemplary embodiment involves a series of spirally wound nitinol sheets 40 nested within one another. The nested spiral sheets 40 serve as a nucleus augmentation, restoring flexibility, elasticity and height to the intervertebral disc. The spiral sheets interact with one another in a fashion similar to a multi-leaf spring. The load is distributed along multiple support spring members, thus reducing the strain on each member. The individual sheets can be varied by thickness, width, spiral diameter, and number to achieve the desired composite spring rate. This feature also offers adjustability to the surgeon for tailoring the implant to each individual disc space. The individual sheets can also be varied in length and spiral construction to achieve the desired final geometric shape, whether it be cylindrical, oval, disc-shaped, or some other geometric configuration. The individual sheets can be wound tighter than their natural state to achieve a reduced profile for insertion via a delivery tool. FIG. 5 shows an individual sheet in the natural state 50 and in the wound or compressed state 52. FIG. 19 shows an exemplary assembly sequence for preparing an individual sheet in its natural state, which includes adjoining two separate nitinol sheets with an inner polymer ply that is fastened in a manner such as laminating or riveting along the interior of the newly combined sheets to prevent separation of the two sheets. FIG. 20 shows a profile reduction sequence for spirally compressing a single sheet in its natural form, such as the single sheet formed by the sequence depicted in FIG. 19. The single sheet in its natural form is forced into a flattened state and the polymer ply used to adjoin the two separate sheets serves as a living hinge. Next, the flattened sheet is rolled into a spiral and the living hinge in the sheet remains flexed. The overall length of the sheet and material thickness will determine the minimum diameter to which the spiral can be wound for profile reduction. The sheets are designed to expand from their compressed state into the outer confines of the nuclear defect. A plurality of such sheets could be nested within one another to create the desired configuration.

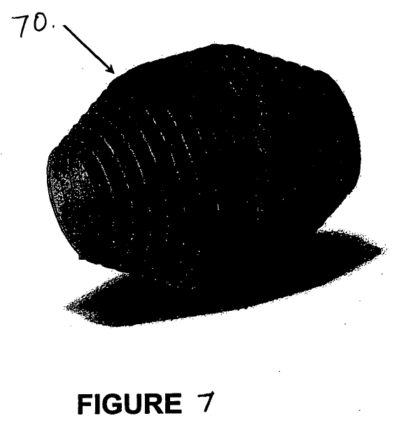

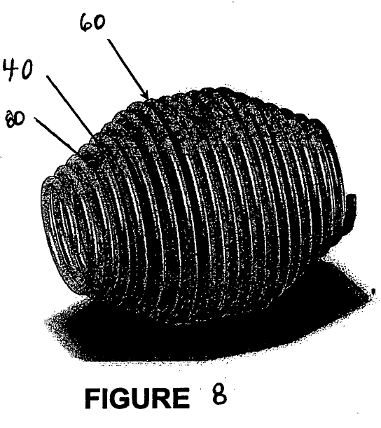

An additional exemplary embodiment that can be used to fill the nuclear defect is a nitinol material that is inserted into the nucleus having a wire construction, and upon expansion, fills the periphery of the nuclear defect. Referring to FIG. 6, a specifically shaped helical wire spring 60 is shown inserted into the nucleus 62. The spring 60 serves as a nucleus augmentation, restoring flexibility, elasticity and height to the vertebral disc. The method of inserting the helical wire spring 60 can be varied. The final outer shape can be varied. The cross-sectional shape of the wire can be varied. For example FIG. 7 shows a spring made of a material fashioned in ribbon form 70.

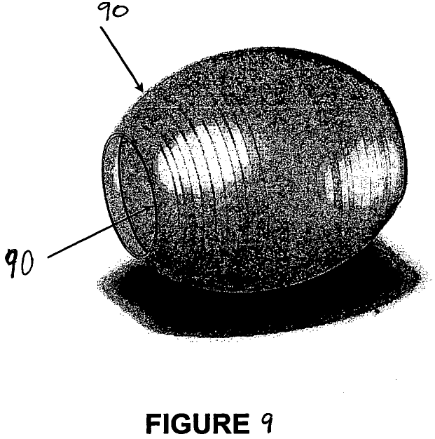

Another exemplary embodiment shown in FIG. 8 is a combination of the exemplary embodiments noted above. In particular, the nested spiral sheets 40 could be situated within the helical spring 60. The helical spring 60 provides the added function of a protective cage, preventing the edges of the sheets 80 from lacerating the annulus wall.

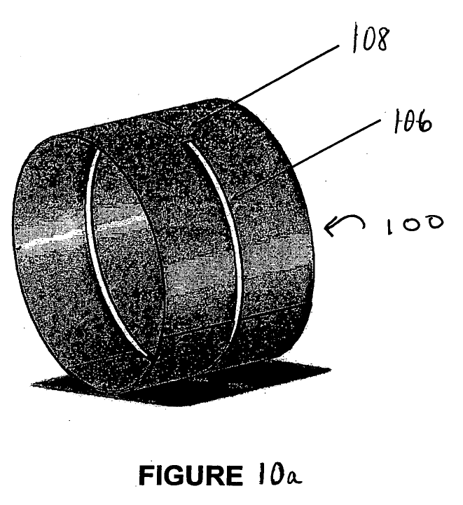

FIG. 9 shows another exemplary embodiment of the present invention, particularly a pliable pouch 90. The pliable pouch 90 is adapted for insertion into a nucleus of a disc, and to receive one or a plurality of implant devices. The pouch can be attached to the distal end of a delivery device cannula. It could also be deployed into the nuclear space prior to the delivery of the primary implant. Further, a fine stainless steel mesh can be molded into the material if wall reinforcement is desired.

As shown in FIGS. 6-8, the final geometric shape of exemplary embodiments of the implant is generally cylindrical and has a non-constant diameter. The diameter of the cylindrical shape is larger near the center of the implant, and smaller at each of the two ends of the implant. In the exemplary embodiments shown, the diameter is largest at or near the center, and then gradually tapers to the smallest diameter at each of the two ends. The diameters at the two ends are shown as being equal, but a combination of different diameters can be used if desired. In addition, the sheets may or may not be porous to allow tissue in-growth.

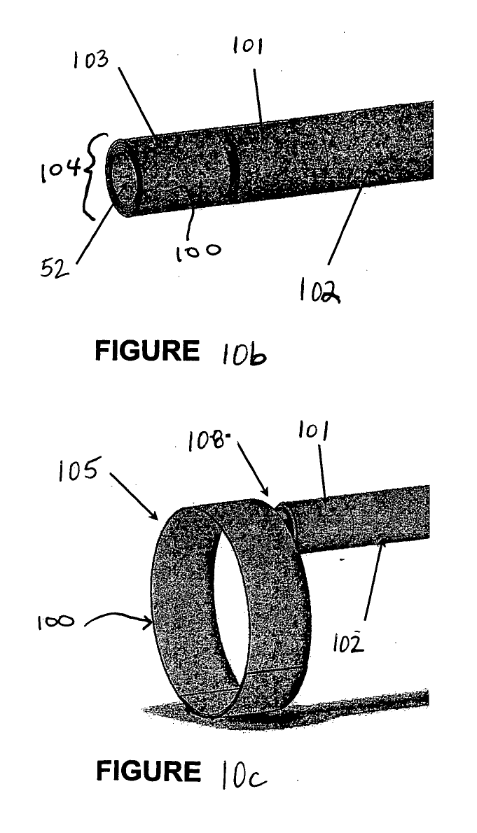

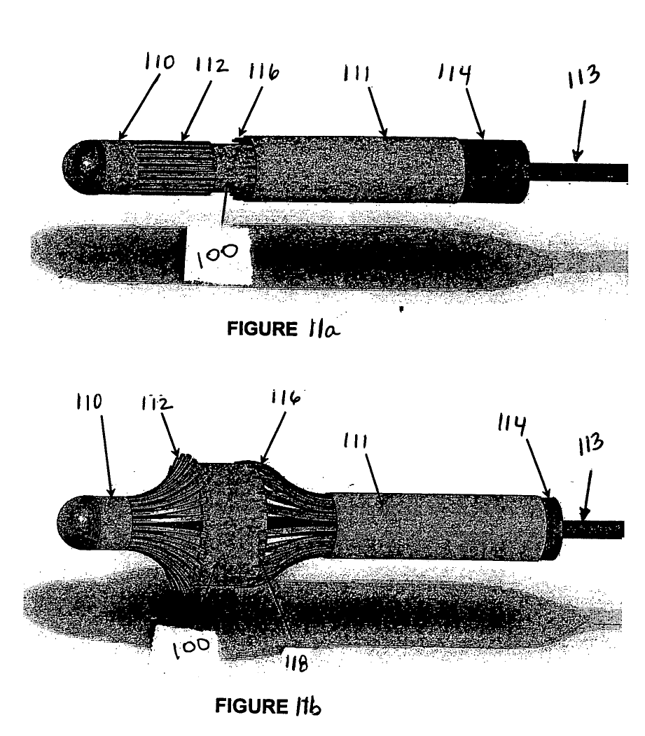

FIGS. 10a, 10b and 10c depict an exemplary method of delivering an implant generally comprising a plurality of “nested spiral sheets” 100 into the nuclear space. The method includes use of an insertion device or delivery cannula 102. The individual sheets 100 are tightly wound to a predetermined diameter 104 and sequentially positioned within the cannula 102, creating a “cartridge” of compressed sheets. Alternatively, each sheet 100 could be deployed individually. As shown in FIG. 10a, each sheet may be configured with slits 106 configured parallel to the opposite ends of the cylindrical shape. The opposing slits 106 from each edge of the sheet are interrupted by a bridge tab 108. As shown in FIG. 10b, the slits 106 and bridge tab 108 generally divide each sheet into a proximal segment 101 and distal segment 103. The sheets are advanced forward by a plunger transferring force through the sequential sheets in the cartridge. As shown in FIG. 10c, the distal segment 103 of the leading sheet deploys from the cannula 102 first, and upon exit, expands to its natural geometric state 105. The proximal sheet 101 still restrained in the cannula 102 provides control of the distal segment 103 for maneuverability and proper positioning of the spiral sheet segment. Upon proper placement, the proximal segment 101 is deployed and the entire sheet is set into place. Each succeeding sheet is deployed in the same manner, resulting in sheets nested one within another. The expandable style sheets may require an expansion tool to deploy properly. The tool may take the form of a balloon or a mechanical linkage mechanism. Every nuclear defect will be different or unique because the amount of nuclear material removed by the surgeon will depend on the condition and presentation of the patient. The expansion tool, such as a balloon, serves to expand the elastic material into the individual boundaries of each defect. Therefore, the same general structure will assume a different shape in each particular defect.

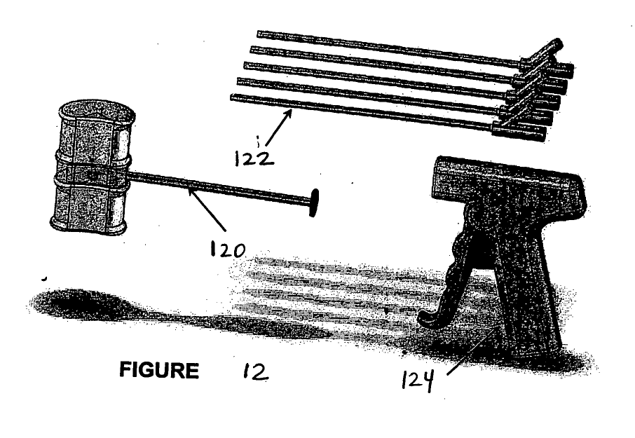

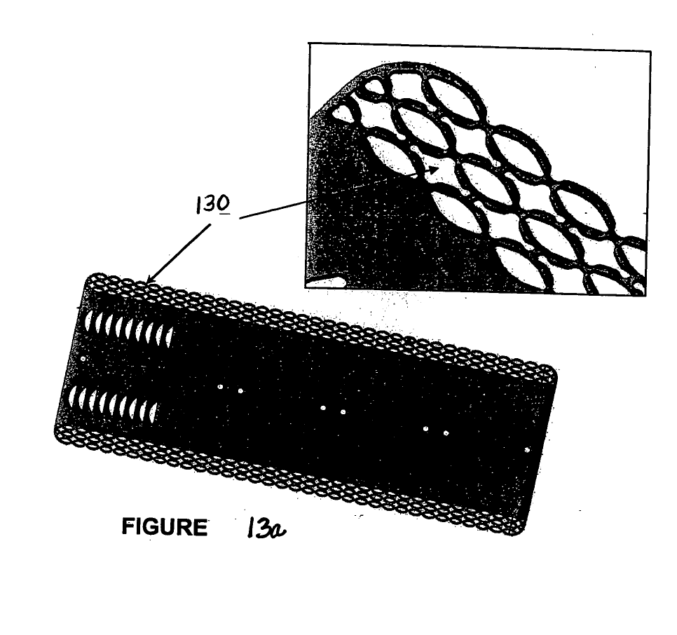

Another exemplary method of delivering the “nested spiral sheets” implant into the nuclear space includes use of a collet mechanism at the distal end of an insertion device as shown in FIGS. 11a and 11b. Like the method described above, the individual sheets 100 are tightly wound and sequentially positioned within the cannula, creating a cartridge of compressed sheets. The sheets are advanced forward by a plunger 113 transferring force through the sequential sheets in the cartridge. A distal collet 110 could be a nitinol tube with protruding fingers 112 that are flexible in a radial fashion. The distal collet 110 offers some radial constraint to the distal end of the leading sheet while also serving as an axial constraint. A proximal collet 114 could also be a nitinol tube with protruding fingers 116 that are flexible in a radial fashion. The protruding fingers 116 are configured such that they capture the outer circumference of the sheet 118 and control radial expansion. An outer sheath 111 engages the proximal collet 114 to control the effective length of the fingers 116. As shown in FIG. 11a, while in the initial position, the outer sheath 111 fully encompasses the fingers 116, and thus fully constrains the sheet in the fully wound state. As shown in FIG. 11b, retracting the outer sheath 111 changes the fulcrum point of the fingers 116, thus allowing them to increase in flexure. Flexure occurs from pressure exerted by the sheet unwinding to its natural state. The distal collet 110 provides a constant axial loading of the sheet toward the proximal collet 114.

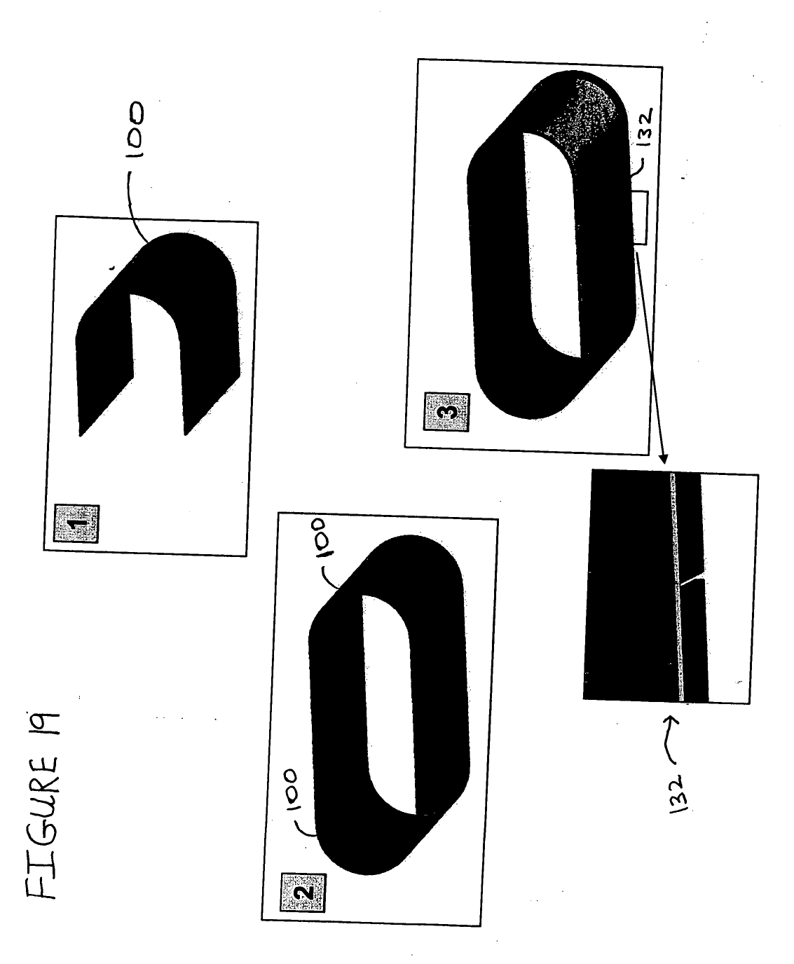

Controlled deployment is achieved by the proper interaction of the distal collet 110, proximal collet 114, and outer sheath 111. Each succeeding sheet can be deployed in the same manner, resulting in sheets nested one within another. Alternatively, only one single sheet in a form of a helix, instead of multiple nested sheets, may be deployed. Another alternative is to place a sheet at a perpendicular axis through the adjacent sheet in order to provide more strength and hydrostatic function. That alternative is shown in FIG. 18, where the inner sheet has been placed at a perpendicular axis through the outer adjacent sheet. To achieve the perpendicular orientation, the sheets or series of sheets would require two access locations or ports. The first port would be used to deploy the first sheets or series of sheets in one orientation, and then the second port would be used to deploy the second sheet or series of sheets in a second orientation that results in the first sheet or series of sheets and second sheet or series of sheets being perpendicular to one another. For example, the first sheet or series could be inserted from a lateral position, and the second series could be inserted from a posterior position. The selection of the particular configuration can be determined by the surgeon on a case-by-case basis depending on the repair that is necessary and/or the personal preference of the surgeon.

Furthermore, with respect to multiple nested sheets being sequentially deployed from the inside, the relative positions of the edges of each sheet can be controlled. More specifically, each sheet can be sequentially deployed randomly, which would allow the edges of each sheet to overlap to whatever degree results from the random deployment. Alternatively, the positioning of the edges of each sheet can be controlled, which would allow for control over the amount and frequency that the edges of the sheets overlap each other, which accordingly, impacts the thickness of the implant, particularly at the locations of the sheet edges. This ability to control these features can be exercised based on the surgeon's discretion and the patient's needs.

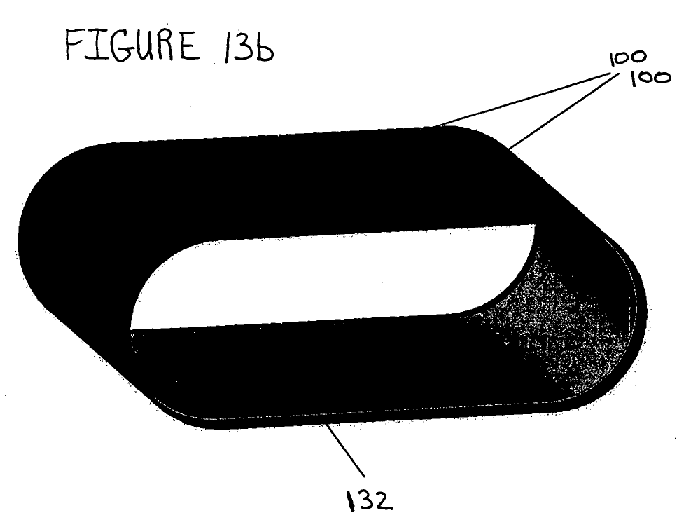

FIG. 12 shows an exemplary embodiment of another delivery device. The delivery device would involve an access cannula 120, several individual cartridges 122, and an actuator handle 124. Each cartridge 122 would house a single crimped sheet 126 and possibly an expander component such as a balloon. The cartridges 122 are capable of being individually mounted into the actuator handle 124 and removed after actuation. The cartridge 122 is capable of being slid through the access cannula and having the implant discharged from the distal end by aid of the actuator handle 124. A pliable pouch could be attached to the distal end of the delivery device cannula. Alternatively, it could be deployed into the nuclear space prior to the delivery of the primary implant.

The embodiment shown in FIG. 12 could be used as follows. First, a guide wire is entered through a small incision toward the treatment site under the guidance of a fluoroscope. Second, dilating tubes are used to achieve proximal placement of the access cannula. Next, under endoscopic visualization, a nerve retractor is used to displace the spinal cord in a safe location. Then an incision of approximately 4 mm in length is made in the annulus, and the access cannula is advanced into the incision. The insertion of the access cannula may require dilating tubes. Next, nucleotomy is performed using graspers or laser technology. Then, a sizer balloon may be inserted and inflated with contrast media. Fluoroscopy can be used to measure nuclear space. If the size of the nuclear space is inadequate, then nucleotomy may be further performed, and the results may be rechecked using the sizer balloon. Next, the pliable implant pouch may be inserted. Then, the nitinol sheet from a first cartridge is deployed. The expansion tool may be used to assist in shaping the sheet in conformance with the size of the nuclear defect. Then, the nitinol sheet from the second cartridge is deployed. Again, the expansion tool may be used to assist in shaping the sheet in conformance with the size of the nuclear defect. The steps of deploying the nitinol sheet and shaping the sheet are repeated until an optimum number of nested sheets have been deployed. Lastly, the delivery tool and access cannula are removed.

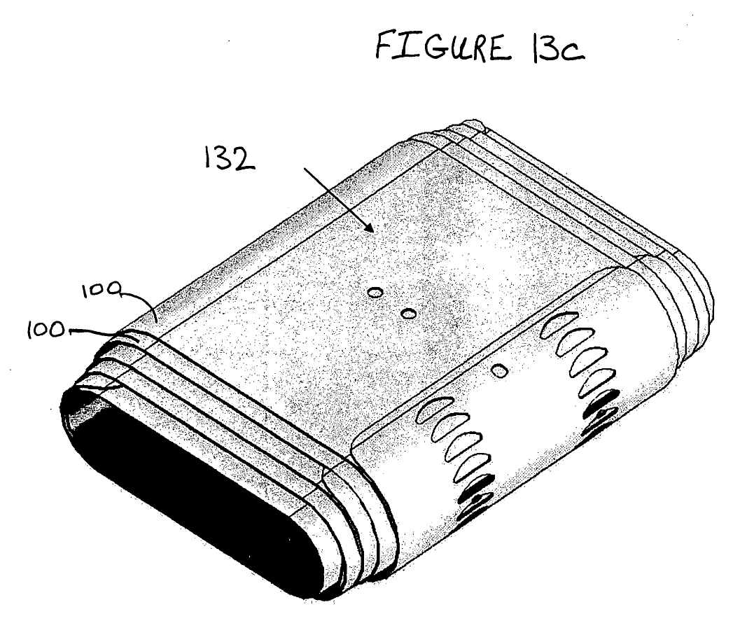

Sharp edges on an implant sheet could lacerate tissue. This is undesirable. Another exemplary embodiment of a sheet is shown in FIG. 13a. The “soft edge” design involves a fabricated edge to reduce edge stiffness, thus eliminating any possible sharp edge. The design to produce a “soft edge” could take many forms, such as the lattice structure 130 shown in FIG. 13a. Other exemplary embodiments utilize a protective surface treatment to round the corners of the edge or to mold, coat, or adhere a polymer rim onto the edge, or a polymer film, coating or sheet layered between nitinol panels. FIG. 13b shows an exemplary nitinol layer having paired nitinol sheets shape-set in cylindrical shape, and the nitinol sheets have a wear prevention barrier 132 that is formed as an inner ply of low friction polymer laminated to the nitinol sheet, which serves a barrier for the next layer of nitinol sheets that would be nested to form a device having multiple nested sheets. FIG. 13c shows an embodiment of multiple nested sheets containing the wear prevention barrier 132. The barrier reduces wear and fretting that results from nitinol on nitinol contact. Another exemplary embodiment is to photo-etch a section along the edge, reducing the material thickness of this section such that it is sufficiently thin to be rolled or folded over, thereby creating a radius along the edge.

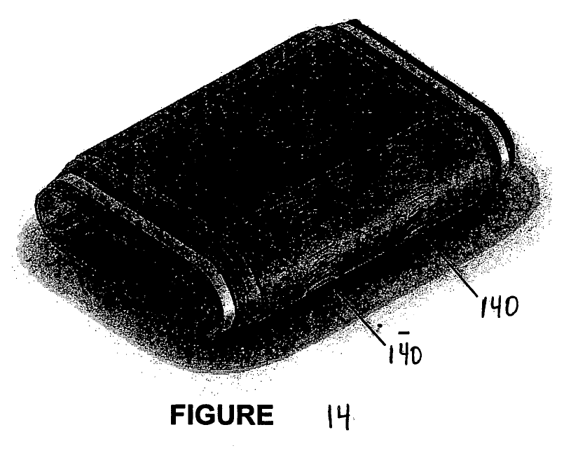

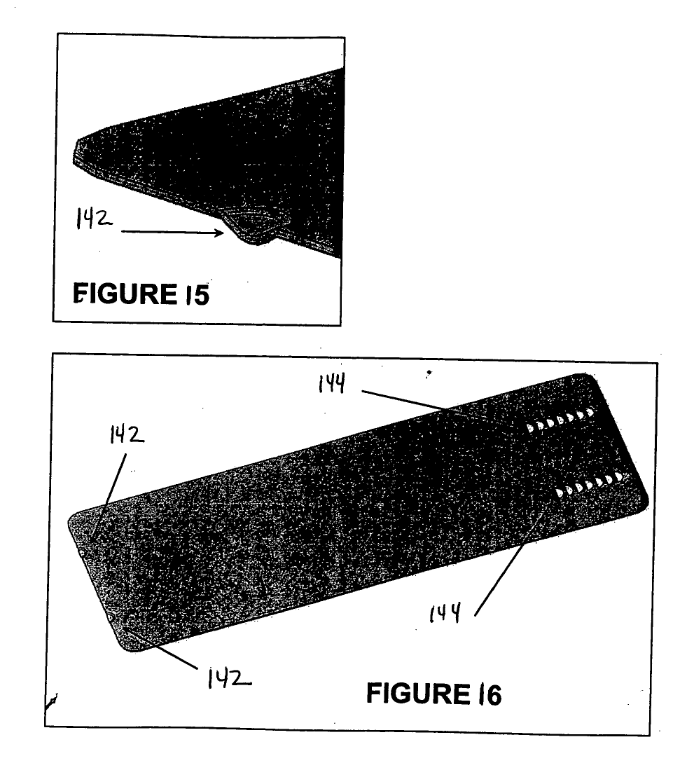

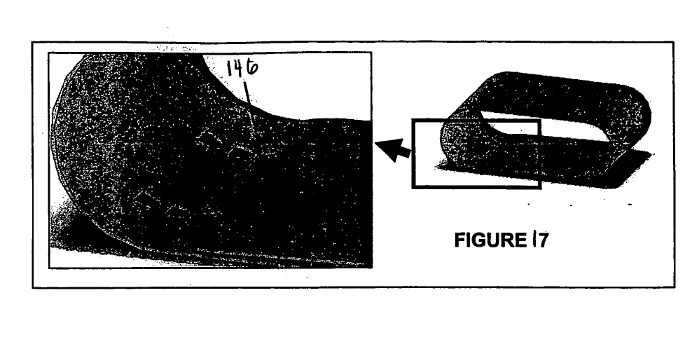

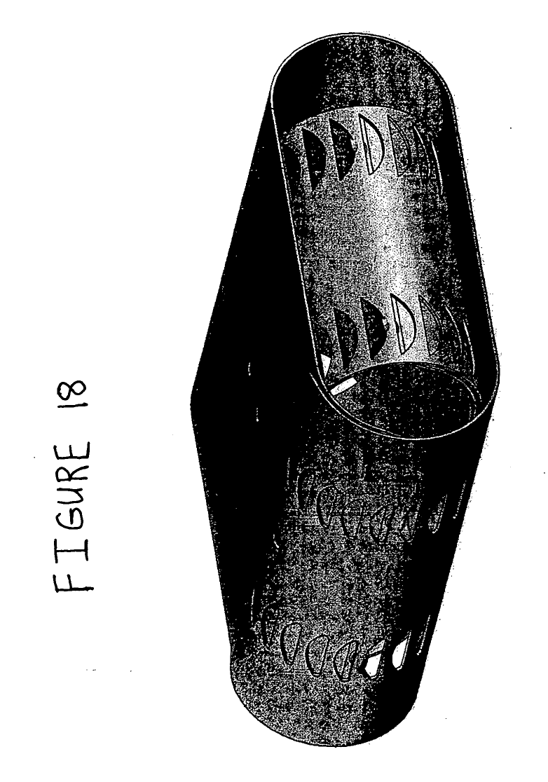

Another exemplary embodiment, as shown in FIG. 14, is to configure the sheets with a ratchet mechanism 140 that involves one or more dimples 142 and mating windows 144 that interlock with one or more selected mating windows 146, as shown in FIGS. 15, 16, and 17. This allows the sheets to be expandable for conforming to the inner disc space. This feature of locking in the expanded state may possibly be achieved by the frictional forces between the overlapping ends. The sheets are also not constrained to a preset shape, thus allowing greater flexibility in terms of spring rates. Conforming to the inner disc space distributes compressive loads across the disc end plates, thus protecting the end plates from excessive strain and possible fractures.

The present implants can also be configured to perform specific functions, or certain aspects of a desired result. For example, with respect to implant embodiments having nested sheets, a plurality of differently configured sheets can be used to configure a single implant, with the individual sheets having a configuration intended to perform a particular function, which contributes to the overall function of the implant. Several functions can be performed by different portions of the present implants, including but not limited to acting as load distributors, neutral zone stiffeners, and/or compressive load bearing members. Several options exist for creating the desired functions through a combination of differently configured sheets. For example, the function of a sheet, or group of sheets, can be impacted by a variety of factors such as the thickness of each sheet, by the placement of the sheet(s) relative to other sheets in the implant, and/or by features such as the sheet(s) being offset-etched or perforated in certain regions. Another possible function of the present implants is to use one or more of the exemplary embodiments to perform a correction-over-time function. For example, the nitinol sheet(s) could be configured to exert a predetermined force in order to gradually restore disc height over time.

In addition to the specific features and embodiments described above, it is understood that the present invention includes all equivalents to the structures and features described herein, and is not to be limited to the disclosed embodiments. For example, the size, shape, and materials used to construct each of the implants can be varied depending on the specific application, as can the methods and devices used to insert them into the patient. Additionally, individuals skilled in the art to which the present expandable implants pertain will understand that variations and modifications to the embodiments described can be used beneficially without departing from the scope of the invention.

What is claimed is:

1. An expandable implant for repair of a defect in a nucleus of an intervertebral disc, the expandable implant comprising:
a pre-insertion shape having a generally cylindrical shape and adapted for insertion into the defect in the nucleus;
a composition that allows the pre-insertion shape to be transformed to a post-insertion shape after the expandable implant is inserted into the defect; and
the post-insertion shape having the generally cylindrical shape and defining a larger volume than the pre-insertion shape, such that the expandable implant substantially fills the periphery of the defect.

2. The expandable implant of claim 1, wherein the pre-insertion shape comprises a spirally wound sheet.

3. The expandable implant of claim 1, wherein the pre-insertion shape comprises a plurality of spirally wound sheets nested within one another.

4. The expandable implant of claim 1, wherein a helical shape is inserted into the defect for receiving the expandable implant.

5. The expandable implant of claim 1, wherein a pliable pouch is inserted into the defect for receiving the expandable implant.

6. The expandable implant of claim 5, wherein at least one portion of the pliable pouch comprises a fine metallic mesh or woven fabric composites.

7. The expandable implant of claim 2, wherein at least one edge of the spirally wound sheet is fabricated to lessen a degree of sharpness of the edge.

8. The expandable implant of claim 7, wherein the fabrication of the edge comprises producing a lattice-like configuration at the edge.

9. The expandable implant of claim 7, wherein the fabrication of the edge comprises adhering a protective material to the edge.

10. The expandable implant of claim 2, wherein the spirally wound sheet comprises one or more dimples and a plurality of mating windows, and wherein each of the one or more dimples interlocks with one of the plurality of mating windows upon the insertion of the spirally wound sheet into the defect and the transformation of the spirally wound sheet.

11. The expandable implant of claim 1, wherein the expandable implant is inserted into the defect via a delivery device.

12. The expandable implant of claim 3, where each of the plurality of spirally wound sheets is individually inserted into the defect via a delivery device, such that the post-insertion shape comprises a nested configuration of the plurality of spirally wound sheets.

13. The expandable implant of claim 11, wherein the delivery device comprises an outer tube into which the expandable implant is inserted and a plunger that drives the inserted implant through the outer tube until the inserted implant is released into the defect.

14. The expandable implant of claim 11, wherein the delivery device comprises an outer sheath coupled with a proximal collet and a distal collet, wherein a default position of the outer sheath is changed in relation to respective positions of the proximal collet and the distal collet in order to insert the expandable implant into the defect.

15. The expandable implant of claim 1, wherein the composition of the expandable implant comprises a shape memory alloy.

16. The expandable implant of claim 15, wherein the shape memory alloy is nitinol.

17. The expandable implant of claim 15 wherein the pre-insertion shape is a non-memory shape that is retained until the expandable implant is activated by temperature or electrical current, such that the activation transforms the expandable implant to a predetermined memory shape that defines the post-insertion shape.

18. The expandable implant of claim 1 wherein the composition of the expandable implant is a superelastic shape memory alloy that changes from the pre-insertion shape to the post-insertion shape automatically after the expandable implant is inserted into the defect.

19. A method of inserting an implant into a defect in a nucleus of an intervertebral disc comprising:
loading the implant into a delivery device adapted for insertion into the defect, wherein the implant is in a compressed form having a generally cylindrical shape;
inserting the delivery device into the intervertebral disc; and
releasing the implant from the delivery device into the defect, wherein the implant transforms from the compressed form to an expanded form having the generally cylindrical shape and designed to generally fill the periphery of the defect.

20. The method of claim 19, wherein the implant comprises a spirally wound sheet.

21. The method of claim 19, wherein the implant comprises a plurality of spirally wound sheets nested within one another.

22. The method of claim 21, wherein each of the plurality of spirally wound sheets are individually loaded into the delivery device and individually released from the delivery device into the defect.

23. The method of claim 20, wherein at least one edge of the spirally wound sheet is fabricated to lessen a degree of sharpness of the edge.

24. The method of claim 20, wherein the spirally wound sheet comprises one or more dimples and a plurality of mating windows, and wherein each of the one or more dimples interlocks with one of the plurality of mating windows upon the releasing of the spirally wound sheet into the defect and the transformation of the spirally wound sheet.

25. The method of claim 19, wherein the delivery device comprises an outer tube into which the implant is loaded and a plunger that drives the loaded implant through the outer tube until the loaded implant is released from the outer tube into the defect.

26. The method of claim 19, wherein the delivery device comprises an outer sheath coupled with a proximal collet and a distal collet, wherein a default position of the outer sheath is changed in relation to respective positions of the proximal collet and the distal collet in order to release the implant into the defect.

27. An expandable implant for repair of a defect in a nucleus of an intervertebral disc, the expandable implant comprising:
a pre-insertion shape having a first generally cylindrical shape and adapted for insertion into the defect in the nucleus;
a composition that allows the pre-insertion shape to be transformed to a post-insertion shape after the expandable implant is inserted into the defect;
the post-insertion shape having a second generally cylindrical shape and defining a larger volume than the pre-insertion shape, such that the expandable implant substantially fills the periphery of the defect; and
wherein the post-insertion shape has a non-constant diameter that is larger near the center of the implant and smaller near each of the two ends of the implant.

28. The expandable implant of claim 27, wherein the pre-insertion shape comprises a spirally wound sheet.

29. The expandable implant of claim 27, wherein the pre-insertion shape comprises a plurality of spirally wound sheets nested within one another.

30. The expandable implant of claim 27, wherein a helical shape is inserted into the defect for receiving the expandable implant.

31. The expandable implant of claim 27, wherein a pliable pouch is inserted into the defect for receiving the expandable implant.

32. The expandable implant of claim 31, wherein at least one portion of the pliable pouch comprises a fine metallic mesh or woven fabric composites.

33. The expandable implant of claim 28, wherein at least one edge of the spirally wound sheet is fabricated to lessen a degree of sharpness of the edge.

34. The expandable implant of claim 33, wherein the fabrication of the edge comprises producing a lattice-like configuration at the edge.

35. The expandable implant of claim 33, wherein the fabrication of the edge comprises adhering a protective material to the edge.

36. The expandable implant of claim 28, wherein the spirally wound sheet comprises one or more dimples and a plurality of mating windows, and wherein each of the one or more dimples interlocks with one of the plurality of mating windows upon the insertion of the spirally wound sheet into the nucleus and the transformation of the spirally wound sheet.

37. The expandable implant of claim 27, wherein the expandable implant is inserted into the defect via a delivery device.

38. The expandable implant of claim 29, where each of the plurality of spirally wound sheets is individually inserted into the defect via a delivery device, such that the post-insertion shape comprises a nested configuration of the plurality of spirally wound sheets.

39. The expandable implant of claim 37, wherein the delivery device comprises an outer tube into which the expandable implant is inserted and a plunger that drives the inserted implant through the outer tube until the inserted implant is released into the defect.

40. The expandable implant of claim 37, wherein the delivery device comprises an outer sheath coupled with a proximal collet and a distal collet, wherein a default position of the outer sheath is changed in relation to respective positions of the proximal collet and the distal collet in order to insert the expandable implant into the defect.

41. The expandable implant of claim 27, wherein the composition of the expandable implant comprises a shape memory alloy.

42. The expandable implant of claim 41, wherein the shape memory alloy is nitinol.

43. The expandable implant of claim 41 wherein the compressed form is a non-memory shape that is retained until the expandable implant is activated by temperature or electrical current, such that the activation transforms the expandable implant to a predetermined memory shape that defines the post-insertion shape.

44. The expandable implant of claim 27 wherein the composition of the expandable implant is a superelastic shape memory alloy that changes from the pre-insertion shape to the post-insertion shape automatically after the expandable implant is inserted into the defect.

45. An expandable implant according to claims 1 or 27, wherein the implant comprises a plurality of spirally wound sheets nested within one another wherein adjacent sheets are oriented parallel to one another.

46. An expandable implant according to claims 1 or 27, wherein the implant comprises a plurality of spirally wound sheets nested within one another wherein adjacent sheets are oriented perpendicular to one another.

47. An expandable implant according to claims 1 or 27, wherein the implant further comprises a wear prevention barrier.

48. The expandable implant of claim 47 wherein the wear prevention barrier comprises a low friction polymer.

49. An expandable implant according to claims 1 or 27, wherein the implant exerts a predetermined force to gradually restore the height of the intervertebral disc.
